(19) United States
(12) Patent Application Publication
(10) Pub. No.: US 2014/0018737 A1
(43) Pub. Date: Jan. 16, 2014

(54) TUNNELER DEVICE WITH INTEGRATED FLUID ASSEMBLY

(71) Applicant: Casey CHEBATOR, Weymouth, MA (US)

(72) Inventor: Casey CHEBATOR, Weymouth, MA (US)

(73) Assignee: TYCO HEALTHCARE GROUP LP, Mansfield, MA (US)

(21) Appl. No.: 13/549,938

(22) Filed: Jul. 16, 2012

(51) Int. Cl. A61M 25/01

(52) U.S. Cl. 604/164.01

(57) ABSTRACT

A tunneler device includes a handle, a shaft extending distally from the handle, a connecting segment, a fluid channel, and a one-way flush valve. The connecting segment includes a body positioned on a distal end of the shaft configured to engage one end of a catheter. The fluid channel extends from the handle to a distal end of the connecting segment. The fluid channel is configured to direct fluid to be selectively passed therethrough for flushing a catheter. The one-way flush valve is supported on the handle and is disposed in fluid communication with the fluid channel.

### BACKGROUND

1. Technical Field

The present disclosure relates to indwelling catheters. More particularly, the present disclosure relates to a tunneler device for preparing a subcutaneous tunnel for catheter placement.

2. Background of Related Art

Catheters are flexible instruments which facilitate the withdrawal and introduction of fluids from and to body cavities, ducts, and vessels. Typically, a distal end of the catheter is implanted into, for example, the vasculature of a patient to withdraw blood from the patient or introduce medicaments into the patient. When a catheter is implanted into the vasculature of a patient, the catheter's distal portion is inserted through an incision in the patient's skin through an opening in the vasculature (venotomy site) until the distal tip is precisely located at a desired site. The proximal portion of the catheter remains external of the patient to provide access to the catheter for infusion and/or withdrawal of fluids such as for hemodialysis. Typically, the proximal portion of the catheter is directed away from the venotomy site to a remote site through a subcutaneous tunnel such that the catheter can be comfortably positioned on a patient's body for long term use and to aid in the prevention of bacterial colonization of the catheter. To accomplish this, a tunneler device is typically secured to the proximal portion of the catheter and the catheter is pulled by the tunneler device through the subcutaneous tunnel.

As can be appreciated, catheters must be properly flushed with a flushing solution, such as a heparinized solution or saline, prior to catheter placement. In known catheters, a separate flush adapter is secured to the proximal end of the catheter to facilitate flushing of the lumens of the catheter. After the flushing procedure is completed, the flush adapter is typically removed from the catheter by cutting the catheter proximate to the flush adapter before the tunneling procedure can commence. The need for an adapter to effect flushing increases the cost of the catheter assembly. In addition, the use of an adapter requires additional steps and therefore increases the duration of the catheter placement procedure. Further, by cutting the flush adapter from the catheter before a tunneling device is attached may cause the flushing fluid to escape the catheter, or the tunneling device may push some flushing fluid out of the catheter, thereby creating an air pocket within the catheter and an increased risk of air embolism to the patient.

It would be beneficial to have a tunneling device that obviates the need for a flush adapter and includes flushing capability for flushing the catheter used in a tunneling procedure to improve the efficiency of the tunneling procedure, and to diminish the chance that flushing fluid will be lost during the procedure.

### SUMMARY

Accordingly, a tunneler device is provided. In general, in one aspect of the present disclosure, a tunneler device includes a handle, a shaft, a connecting segment, and a flush assembly. The shaft extends distally from the handle. The connecting segment is positioned on a distal end of the shaft. The connecting segment is configured to engage one end of a catheter. The flush assembly includes a fluid channel and a one-way flush valve. The flush valve is supported on the handle and is disposed in fluid communication with the fluid channel. The flush valve and the fluid channel are configured to facilitate a supply of a flush solution from the handle to the connecting segment and into the catheter. The flush valve may include a luer adapter configured to releasably engage a medical device.

The tunneler device may include a tunneling cap enclosing the connecting segment, wherein the connecting segment includes an extension segment that is configured to engage the end of the catheter. The fluid channel extends to an opening defined in a distal end of the extension segment. The cap is selectively removable from the connecting segment to expose the extension segment. The cap is configured to facilitate passage of the tunneler device through subcutaneous tissue during a tunneling procedure.

The extension segment may include a first extension segment and a second extension segment. The fluid channel may include a first branch and a second branch defined within the first and second extension segments, respectively. The first extension segment is configured to be received within the first lumen of a multilumen catheter and the second extension segment is configured to be received within the second lumen of the multilumen catheter. The first branch extends to a first opening defined in the first extension segment and the second branch extends to a second opening defined in the second extension segment. In various embodiments, the multilumen catheter may include two, three, four, or more lumens, and the connecting segment may include an equal or lesser number of extension segments.

According to another aspect of the present disclosure, a tunneler device includes a handle, a shaft extending distally from the handle, a connecting segment, a fluid channel, and a one-way flush valve. The connecting segment includes a body positioned on a distal end of the shaft configured to engage one end of a catheter. The fluid channel extends from the handle to a distal end of the connecting segment. The fluid channel is configured to direct fluid to be selectively passed therethrough for flushing a catheter. The one-way flush valve is supported on the handle and is disposed in fluid communication with the fluid channel. The one-way flush valve may include a luer adapter configured to releasably engage a medical device.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a side view of one embodiment of a tunneler device according to the present disclosure connected to a fluid source;

FIG. 2 is an enlarged side view of a distal region of the presently disclosed tunneler device, with a cap being shown separated from a connecting segment of the presently disclosed tunneler device;

FIG. 3 is an enlarged side view of the distal region of the presently disclosed tunneler device, with the connecting segment of the presently disclosed tunneler device shown inserted into one end of a lumen of a catheter;

FIG. 4 is an enlarged side view of the distal region of another embodiment of the presently disclosed tunneler device and another catheter with dual lumens; and

FIG. 5 is an enlarged side view of a proximal region of the presently disclosed tunneler device with a syringe shown engaged with a flush valve of the presently disclosed tunneler device.

### DETAILED DESCRIPTION

The embodiments of the present disclosure are directed to a tunneling system including a tunneling device capable of facilitating flushing of a catheter as well as subcutaneous catheter placement. The tunneling system of the present disclosure may have various medical applications. For example, during a hemodialysis catheter implantation procedure, the tunneling device may be manipulated by a clinician to create or enlarge a subcutaneous tunnel within a patient to properly position the catheter on the patient. It is envisioned that the presently disclosed tunneling system may be employed in any other suitable procedure.

As used herein, the term “clinician” refers to a doctor, a nurse, or any other care provider and may include support personnel. The term “proximal” refers to the portion of a structure that is closer to a clinician, whereas the term “distal” refers to the portion that is farther from the clinician.

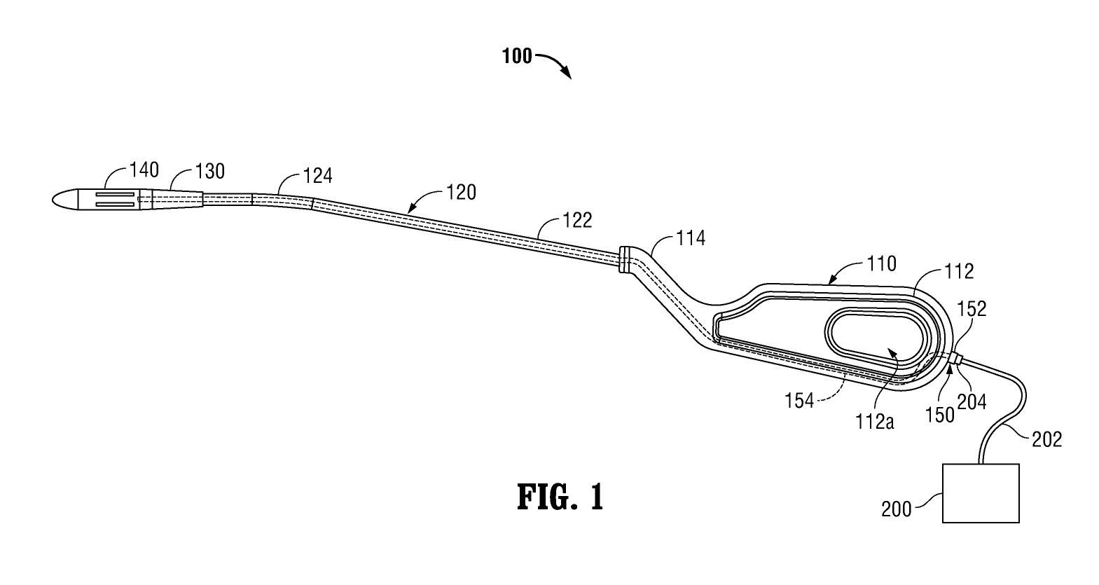

With reference to FIG. 1, a tunneler device according to the present disclosure is shown generally as tunneler device 100. Although tunneler device 100 will be described for use in the placement of a dialysis catheter within a patient, tunneler device 100 may be used for the placement of various other medical implements. Tunneler device 100 includes a handle 110, a shaft 120 extending from handle 110, a connecting segment 130 mounted on a distal end of shaft 120, a cap 140 selectively engageable with the connecting segment 130, and a flush assembly 150. Any or all of the components of tunneler device 100 may be sterilizable, and thus, reusable. Alternatively, tunneler device 100 may be disposable.

With continuing reference to FIG. 1, handle 110 is supported on a proximal end of tunneler device 100 and is configured for operable engagement by a clinician. In particular, handle 110 may include a loop 112 at a proximal end of handle 110 which defines an opening 112a that is dimensioned to receive a clinician's fingers. A distal handle segment 114 is provided at a distal end of handle 110 that supports a proximal end of shaft 120. Distal handle segment 114 may be obliquely arranged with respect to shaft 120 at any suitable angle to displace or offset the hand of the clinician relative to the patient during use of tunneler device 100 in a tunneling procedure. Handle 110 may include ridges, bumps, knurls, and/or any other suitable non-slip features configured to improve gripping engagement of handle 110 by a clinician. Handle 110 may also be configured for operable engagement with a robotic arm (not shown) or other mechanical manipulating device.

A proximal end of shaft 120 is secured to, and may be embedded within, distal handle segment 114 of handle 110 to connect handle 110 and shaft 120 together. Shaft 120 extends distally from a distal end of handle 110 and includes a proximal shaft segment 122 and a distal shaft segment 124. In one embodiment, handle 110 is molded about a proximal end of proximal shaft segment 122 of shaft 120. Distal shaft segment 124 may be obliquely arranged with respect to proximal shaft segment 122 at any suitable angle to facilitate formation of a tunnel in a patient as desired. Further, shaft 120 may be malleable to enable the clinician to bend shaft 120 as needed for a particular procedure. For example, the clinician may bend shaft 120 into a curved shape to enable tunneler device 100 to create a curved or arced tunnel.

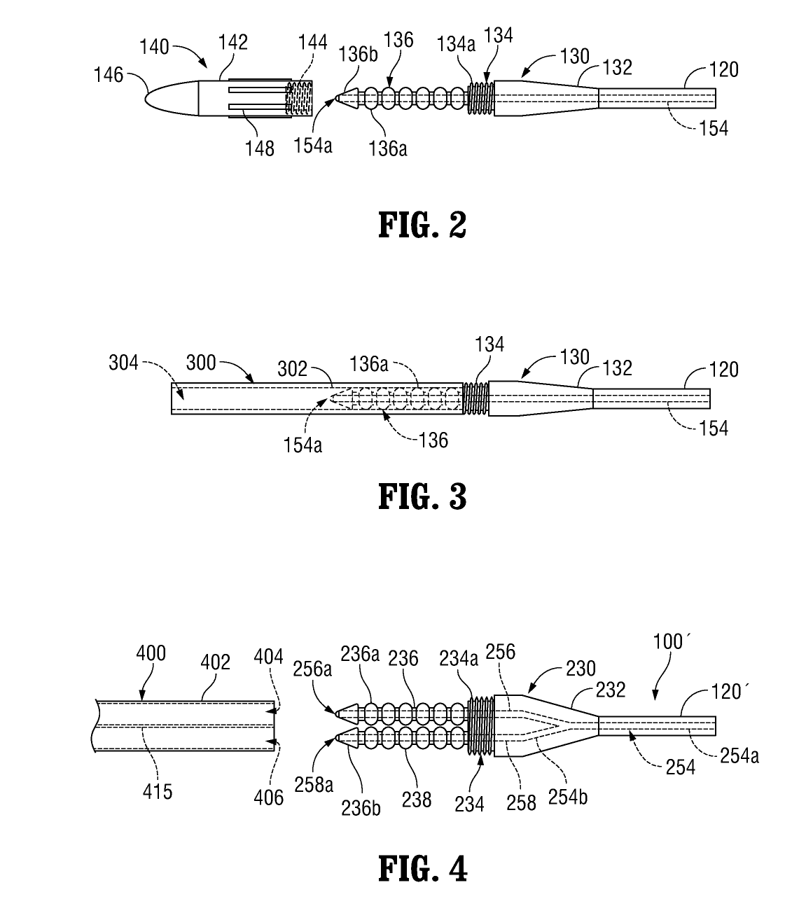

Turning now to FIG. 2, connecting segment 130 is supported on the distal end of shaft 120 and includes main body 132 for supporting connecting segment 130 on the distal end of shaft 120, a threaded segment 134 for releasably engaging a tunneling cap 140, and an extension segment 136 for frictionally engaging one end of a catheter such as catheter 300 or catheter 400 (see FIGS. 3-4). Threaded segment 134 includes one or more external threads 134a and may be positioned adjacent main body 132 to facilitate a threaded engagement with internal threads 144 of tunneling cap 140. Extension segment 136 extends distally from a distal end of threaded segment 134 and may include a plurality of barbs 136a and a blunt tip 136b. Although illustrated as spherically shaped barbs 136a, other barb configurations are envisioned. Extension segment 136 is configured and dimensioned to be positioned within a lumen of catheter 300 when tunneling cap 140 is removed from connecting segment 130 such that barbs 136a frictionally engage inner surfaces of catheter 300 to secure extension segment 136 within catheter 300. As may be appreciated, connecting segment 130 may be integrally formed with shaft 120, or, in the alternative, secured to shaft 120 using known fastening techniques such as overmolding.

Referring again to FIG. 2, tunneling cap 140, which may operate as a tissue dissector or expander, may include a substantially bullet-shaped body 142 that facilitates passage through subcutaneous tissue during a tunneling procedure. Body 142 includes internal threads 144 at a proximal end and a substantially rounded atraumatic tip 146 at a distal end. As discussed above, internal threads 144 of body 142 mate with external threads 134a of threaded segment 134 of connecting segment 130 to releasably secure tunneling cap 140 to connecting segment 130. With tunneling cap 140 secured to connecting segment 130, extension segment 136 is enclosed within tunneling cap 140 to facilitate passage of tunneler device 100 through subcutaneous tissue. Although tunneling cap 140 and connecting segment 130 are configured for threaded engagement with each other, any suitable arrangement may be utilized to releasably secure tunneling cap 140 to connecting segment 130. For example, tunneling cap 140 may be secured to connecting segment 130 with a bayonet coupling or via friction fit arrangement. Tunneling cap 140 may be provided in various sizes for creating tunnels of different sizes. In this manner, tunneler device 100 may be used for the implantation of catheters having different sizes. Tunneling cap 140 may include longitudinal ribs 148 which extend along at least a portion of a length of body 142. Ribs 148 may be configured to facilitate manipulation of tunneling cap 140 by a clinician during securement and removal of tunneling cap 140 to and from connecting segment 130.

As best depicted in FIG. 1, flush assembly 150 includes a flush valve 152 supported on handle 110 and a fluid channel 154 defined within tunneler device 100 and extending from flush valve 152 to a distal end of connecting segment 130. Fluid channel 154 is dimensioned to enable fluid to be passed through the tunneler device 100 from flush valve 152 to a distal end of extension segment 136. Fluid may be passed through fluid channel 154 for flushing catheter 300 (see FIG. 3) prior to, or subsequent to a tunneling procedure. In one embodiment, fluid channel 154 is defined by a tube (not shown) which is enclosed within handle 110, shaft 120, and connecting segment 130 of tunneler device 100. Alternatively, fluid channel 154 can be defined directly by the handle 110, shaft 120, and/or connecting segment 130 of tunneler device 100.

With regard to FIGS. 2-3, fluid channel 154 defines an opening 154a at the distal end of extension segment 136 of connecting segment 130. Opening 154a is disposed in fluid communication with a lumen 304 extending through a body 302 of catheter 300 when extension segment 136 is inserted within lumen 304 of catheter 300. The opening 154a may be defined anywhere along the extension segment 136 of connecting segment 130 that will enable fluid passage through the tunneling device 100 into the lumen 304 of catheter 300 to facilitate flushing of catheter lumen 304. The opening 154a may include a valve such as a one-way valve (not shown) to seal a distal end of fluid channel 154.

In alternate embodiments, as shown in FIG. 4, tunneler device 100′ includes a connecting segment 230 which is supported on the distal end of shaft 120′ and includes main body 232 for supporting connecting segment 230 on the distal end of shaft 120′, a threaded segment 234 for engaging the tunneling cap 140 (FIG. 2), a first extension segment 236 dimensioned to be frictionally received within a first lumen 404 of catheter 400, and a second extension segment 238 dimensioned to be frictionally received within a second lumen 406 of catheter 400. Threaded segment 234 includes external threads 234a and may be positioned adjacent main body 232 for releasably engaging internal threads 144 of tunneling cap 140 as discussed above with respect to threaded segment 134.

Extension segments 236 and 238 extend distally from a distal end of threaded segment 234 in parallel relationship. Each extension segment 236, 238 may include a plurality of barbs 236a and a blunt tip 236b. Extension segments 236, 238 are dimensioned to be respectively positioned within first and second lumens 404, 406 of catheter 400. In this position, barbs 236a frictionally engage inner surfaces of catheter 400, for example, a catheter septum 415, to secure extension segments 236, 238 within catheter 400 when tunneling cap 140 is removed from connecting segment 230, as discussed in greater detail below. As may be appreciated, connecting segment 230 may be integrally formed with shaft 120, or, in the alternative, secured to shaft 120′ using known fastening techniques such as overmolding.

Connecting segment 230 of main body 232 defines a fluid channel 254 therethrough that enables a simultaneous flushing of first and second lumens 404, 406 of a catheter 400. Fluid channel 254 includes a single channel portion 254a that transitions to a multichannel portion 254b to permit fluid to pass through tunneling device 100′ for flushing first and second lumens 404, 406 of catheter 400 when first and second extension segments 236, 238 are secured within first and second lumens 404, 406, respectively, of catheter 400. In particular, multichannel portion 254b includes a first branch 256 and a second branch 258 which extend from the single channel portion 254a through extension segments 236, 238. First branch 256 extends to a first opening 256a defined within first extension segment 236. Similarly, second branch 258 extends to a second opening 258a defined within second extension segment 238. Like opening 154a, first and second openings 256a and 258a may include a valve such as a one-way valve (not shown) to seal distal ends of first and second branches 256, 258 of fluid channel 254.

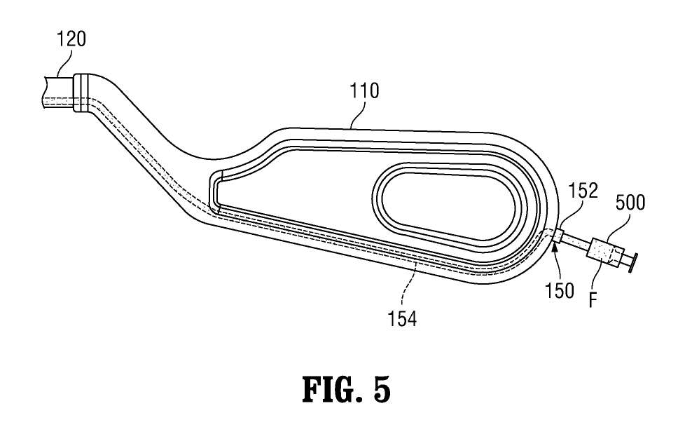

Referring to FIGS. 1 and 5, a luer adapter may be provided about flush valve 152 to facilitate securement of a medical device, such as flushing device 200 (FIG. 1) or syringe 500 (FIG. 5), to tunneler device 100. Notably, syringe 500, which may be pre-filled, can be inserted into flush valve 152 so that fluid “F”, such as heparinized solution or saline, stored within syringe 500 may be supplied through fluid channel 154 into a lumen of catheter 300 or catheter 400 upon the actuation of the syringe 500 for flushing catheter 300 or catheter 400. Flush valve 152 defines a one-way valve (not shown) which enables fluid to flow from the medical device through flush valve 152 into fluid channel 154 but prevents fluid from flowing from fluid channel 154 through flush valve 152. By providing a one-way valve on tunneler device 100, flush solution which has been supplied to a catheter through flush valve 152 will not drain from the catheter while the catheter is attached to the tunneler device 100. Any suitable independent flushing device 200 may be operatively coupled to flush valve 152 (see FIG. 1) to inject fluids into fluid channel 154 to effect flushing of a catheter. Flushing device 200 may include one or more tubes 202 and one or more couplings 204 that are securable to flush valve 152 via a luer connector to establish fluid communication between flushing device 200 and tunneler device 100.

In use, the distal end of tunneler device 100, with tunneling cap 140 attached, is inserted through an incision positioned remote from a venotomy site of a patient and pushed through the patient's tissue to a location positioned adjacent the venotomy site to create a subcutaneous tunnel. The distal end of the tunneler 100 is positioned to extend from the subcutaneous tunnel positioned adjacent to the venotomy site. Next, cap 140 is removed from the distal end of the tunneler device 100 to expose connecting assembly 130, 230 for insertion into the proximal end of catheter 300, 400. Then, with the tunneler device 100 connected to the respective catheter 300, 400, tunneler device 100 may be reverse tunneled back through the subcutaneous tunnel. However, prior to insertion of the distal end of catheter 300, 400 into the patient, lumens 304, 404, 406 of catheters 300, 400 are flushed with a flushing solution which is injected through flush valve 152 and fluid channel 154, 254 of flushing assembly 150 of tunneler device 100, 100′. The distal end of the catheter 300, 400 may be inserted into the patient either before or after pulling the proximal end of the catheter 300, 400 through the created subcutaneous tunnel. In either procedure, with the proximal end of catheter 300, 400 drawn through the subcutaneous tunnel after being reverse tunneled, the proximal end of catheter 300, 400 is clamped and cut to disengage the tunneler device 100, 100′ from the rest of catheter 300, 400.

As can be appreciated, the tunneler device of the present disclosure and any of its components may be formed of any suitable polymeric or metallic material and combinations thereof.

Persons skilled in the art will understand that the structures and methods specifically described herein and illustrated in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, it is envisioned that the elements and features illustrated or described in connection with one exemplary embodiment may be combined with the elements and features of another without departing from the scope of the present disclosure, and that such modifications and variations are also intended to be included within the scope of the present disclosure. Accordingly, the subject matter of the present disclosure is not to be limited by what has been particularly shown and described.

1. A tunneler device comprising:
a handle;
a shaft extending distally from the handle;
a connecting segment positioned on a distal end of the shaft, the connecting segment being configured to engage one end of a catheter; and
a flush assembly including a fluid channel and a one-way flush valve, the flush valve being supported on the handle and being disposed in fluid communication with the fluid channel, the flush valve and the fluid channel being configured to facilitate a supply of a flush solution from the handle to the connecting segment and into the catheter.

2. The tunneler device of claim 1, wherein the flush valve includes a luer adapter configured to releasably engage a medical device.

3. The tunneler device of claim 1, further comprising a tunneling cap enclosing the connecting segment, wherein the connecting segment includes an extension segment that is configured to engage the end of the catheter, the cap being selectively removable from the connecting segment to expose the extension segment, the cap being configured to facilitate passage of the tunneler device through subcutaneous tissue during a tunneling procedure.

4. The tunneler device of claim 3, wherein the fluid channel extends to an opening defined in a distal end of the extension segment.

5. The tunneler device of claim 3, wherein the extension segment includes a first extension segment and a second extension segment and the fluid channel includes a first branch defined within the first extension segment and a second branch defined within the second extension segment.

6. The tunneler device of claim 5, wherein the first extension segment is configured to be received within the first lumen of a multilumen catheter and the second extension segment is configured to be received within the second lumen of the multilumen catheter.

7. The tunneler device of claim 6, wherein the first branch extends to a first opening defined in the first extension segment and the second branch extends to a second opening defined in the second extension segment.

8. A tunneler device comprising:
a handle;
a shaft extending distally from the handle;
a connecting segment including a body positioned on a distal end of the shaft configured to engage one end of a catheter;
a fluid channel extending from the handle to a distal end of the connecting segment, the fluid channel configured to direct fluid to be selectively passed therethrough for flushing a catheter; and
a one-way flush valve supported on the handle and being disposed in fluid communication with the fluid channel.

9. The tunneler device of claim 8, wherein the one-way flush valve includes a luer adapter configured to releasably engage a medical device.

10. The tunneler device of claim 8, further comprising a tunneling cap enclosing the connecting segment, wherein the connecting segment includes an extension segment that is configured to engage the end of the catheter, the cap being selectively removable from the connecting segment to expose the extension segment, the cap being configured to facilitate passage of the tunneler device through subcutaneous tissue during a tunneling procedure.

11. The tunneler device of claim 10, wherein the fluid channel extends to an opening defined in a distal end of the extension segment.

12. The tunneler device of claim 10, wherein the extension segment includes a first extension segment and a second extension segment and the fluid channel includes a first branch defined within the first extension segment and a second branch defined within the second extension segment.

13. The tunneler device of claim 12, wherein the first extension segment is configured to be received within the first lumen of a multilumen catheter and the second extension segment is configured to be received within the second lumen of the multilumen catheter.

14. The tunneler device of claim 13, wherein the first branch extends to a first opening defined in the first extension segment and the second branch extends to a second opening defined in the second extension segment.

15. A tunneler device comprising:
a handle;
a shaft extending distally from the handle;
a connecting segment positioned on a distal end of the shaft configured to engage one end of a catheter; and
a flush assembly defining a fluid channel and supporting a one-way flush valve, the flush valve being supported on an outer surface of the handle and being disposed in fluid communication with the fluid channel, the fluid channel extending from the flush valve to the connecting segment to facilitate a supply of a flush solution from the handle to the connecting segment.

16. The tunneler device of claim 15, wherein the handle includes a loop that defines an opening dimensioned to receive a clinician's fingers.

17. The tunneler device of claim 8, wherein the one-way flush valve is supported on an outer surface of the handle.

18. The tunneler device of claim 8, wherein the handle includes a loop that defines an opening dimensioned to receive a clinician's fingers.

19. The tunneler device of claim 18, wherein the opening of the handle extends through opposed outer side surfaces of the handle.

20. The tunneler device of claim 1, wherein the flush valve is supported on an outer surface of the handle.
